(19) United States
(12) Patent Application Publication
(10) Pub. No.: US 2026/0108699 A1
(43) Pub. Date: Apr. 23, 2026

(54) NOVEL CLOSED-LOOP FEEDBACK NEUROMODULATION SYSTEM FOR IMPROVING SLEEP

(71) Applicant: SYNAPTIC HEALTH LLC, Saint Paul, MN (US)

(72) Inventors: Benjamin Arcand, Minneapolis, MN (US); Bryce C Beverlin, II, Saint Paul, MN (US); Brian Krohn, New Brighton, MN (US)

(73) Assignee: SYNAPTIC HEALTH LLC, Saint Paul, MN (US)

(21) Appl. No.: 19/353,503

(22) Filed: Oct. 8, 2025

Related U.S. Application Data
(60) Provisional application No. 63/704,977, filed on Oct. 8, 2024.

(51) Int. Cl. A61M 21/02; A61F 9/04; A61M 21/00; G16H 50/70

(52) U.S. Cl. CPC A61M 21/02; A61F 9/04; A61M 2021/0066; A61M 2205/3673; A61M 2230/10; A61M 2230/50; G16H 50/70

(57) ABSTRACT

A gadget smart sleeping mask with artificial intelligence machines for configuring, through a human machine interface, a closed loop neuromodulation feedback module for improving sleeping, including a thermoregulation member configured for thermoregulation of facial nerves for enhancing healthy sleeping time. EEG electrode sensors are connected to the thermoregulation member; the EEG sensors are placed along forehead part of the mask for contacting forehead skin to measure human brain activities according to indices of the healthy sleeping time. A processor is connected to each of the thermoregulation member and the plurality of the EEG electrodes; the processor classifies sleep stages according to real time computing data and modules. A thermocouple heating and cooling component is connected to each of the thermoregulation member, the plurality of the EEG electrode sensors, and the processor for delivering temperature stimulation to skin of wearer of the mask; the thermocouple heating and cooling component is disposed within a physical measurement of boundaries of the sleep mask area after a user wears the sleep mask. A computing and electronic gadgets workbench member is connected to each of the thermoregulation member, the plurality of the EEG electrode sensors, the thermocouple heating and cooling component, and the processor; a graphical user interface is configured for healthy sleep stages.

### CROSS-REFERENCE TO RELATED APPLICATIONS

N/A

### BACKGROUND OF THE INVENTION

### 1. Field of the Invention

The disclosure generally relates to a healthier sleeping technique improving mechanism but more particularly to a closed loop neuromodulation system and method configured by artificial intelligence for improving sleeping

### 2. Description of Related Art

Conventional health related electronic and computing gadget systems and methods lack real time optimization capabilities regarding the artificial intelligence systems in the modern societies. There is a need for innovative solutions that combine advanced technologies, such as artificial intelligence, machine learning, and augmented reality, for improving sleeping techniques. Alzheimer's disease (AD) is a progressive debilitating disease and the most prevalent cause of dementia in the United States. The Centers for Disease Control and Prevention (CDC) estimates that 6 million Americans have AD, with a projected increase to 14 million by 2060. The total estimated US healthcare costs for the treatment of AD in 2020 are estimated at $305 billion each year and are expected to increase to $1 trillion by 2050. One estimate suggests that delaying the onset of symptomatic AD by 5 years would reduce the total economic burden of AD by 43% in 2050. Thus, slowing the progression of AD could save hundreds of billions of dollars in healthcare costs. Several high-profile drugs have attempted to slow the progression of AD by disrupting the accumulation of pathogenic proteins, specifically amyloid beta (AB), which build up in the brain as AD worsens. Any pharmaceutical approach is likely to be invasive, expensive, and have significant side-effects. The recently approved and controversial drug aducanumab failed to demonstrate significantly improved cognition, is an invasive intravenous infusion, costs $56,000 per year per person, and 40% of clinical trial participants experienced brain swelling. The controversial approval of aducanumab suggests that regulators are desperate for a solution, but given the drug's failure to demonstrate clear efficacy and affordability, other approaches should be considered.

Like alternative to pharmaceuticals, there is increasing evidence that naturally promoting healthy sleep architecture, specifically slow wave sleep (SWS), could slow or reverse the progression of AD through enhanced glymphatic system clearance. The glymphatic system is the natural bio-mechanical process that removes waste from the brain, including Aß accumulations, tau depositions, and other brain toxins. The glymphatic system is most active during sleep and in particular during SWS when neurons contract, thereby increasing intracellular space and allowing for increased flow of cerebrospinal fluid and the removal of pathogenic proteins and other toxins. Experiments have shown that disrupting SWS also disrupts glymphatic clearance and results in an increase of pathogenic proteins. On the other hand, reinitiating a healthy SWS pattern halts the accumulation of pathogenic proteins38. Therefore, promoting SWS improves an active glymphatic system and the removal of pathogenic proteins and is a promising approach to slowing the progression of AD. However, currently there are no approved therapies that boost the glymphatic clearance system for AD patients and patients at high risk for AD. Thus, there is a significant unmet clinical need to develop a non-invasive and affordable device that promotes glymphatic clearance for pre-AD and early-stage AD patients. Thus, there is a requirement of improving sleeping techniques.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later. The present invention provides a system for a gadget smart sleeping mask collaborating with artificial intelligence machines for configuring, through a human machine interface, a closed loop neuromodulation feedback module for improving sleeping, the system includes a thermoregulation member configured for thermoregulation of facial nerves for enhancing healthy sleeping time. A plurality of EEG electrode sensors is connected to the thermoregulation member; wherein the EEG sensors are placed along forehead part of the mask for contacting forehead skin to measure human brain activities according to indices of the healthy sleeping time. A processor is connected to the thermoregulation member and the plurality of the EEG electrodes; wherein the processor classifies sleep stages according to real time computing data and modules. A thermocouple heating and cooling component is connected to the thermoregulation member, the plurality of the EEG electrode sensors, and the processor for delivering temperature stimulation to skin of wearer of the mask; wherein the thermocouple heating and cooling component is disposed within a physical measurement of boundaries of the sleep mask area after a user wears the sleep mask. A computing and electronic gadgets workbench member is connected to the thermoregulation member, the plurality of the EEG electrode sensors, the thermocouple heating and cooling component, and the processor; wherein a graphical user interface is configured for healthy sleep stages. A front region of the sleeping mask covers eyes and region around the eyes; and wherein the mask is fastened, to head of the mask wearer, by a strap around the head, at a back region of the sleeping mask.

The sleeping mask extends above eyebrows and till hairline of the mask wearer and down below till nostrils of the mask wearer. The thermocouple heating and cooling component comprising a peltier device. The sleeping mask is configured as a sleep wearable medical device. A temperature changes module configured to measure temperature changes on skin of face of the mask wearer by affecting autonomous nervous system and brain.

The present invention provides a gadget smart sleeping mask device collaborating with artificial intelligence machines for configuring, through a human machine interface, a closed loop neuromodulation feedback module for improving sleeping, the device including a thermoregulation member configured for thermoregulation of facial nerves for enhancing healthy sleeping time. A plurality of EEG electrode sensors is connected to the thermoregulation member; wherein the EEG sensors are placed along forehead part of the mask for contacting forehead skin to measure human brain activities according to indices of the healthy sleeping time. A processor is connected to the thermoregulation member and the plurality of the EEG electrodes; wherein the processor classifies sleep stages according to real time computing data and modules. A thermocouple heating and cooling component is connected to the thermoregulation member, the plurality of the EEG electrode sensors, and the processor for delivering temperature stimulation to skin of wearer of the mask; wherein the thermocouple heating and cooling component is disposed within a physical measurement of boundaries of the sleep mask area after a user wears the sleep mask. A computing and electronic gadgets workbench member is connected to the thermoregulation member, the plurality of the EEG electrode sensors, the thermocouple heating and cooling component, and the processor; wherein a graphical user interface is configured for healthy sleep stages.

The present invention provides a computer implemented method for a sleeping mask device that configures, through a human machine interface, a closed loop neuromodulation feedback module for improving sleeping, the method including configuring, by the at least one processor and at the least one memory, at least one of a mobile phone, a laptop, an electronic device, a personal digital assistant, a robotic device, for respective thermoregulation of facial nerves for enhancing healthy sleeping time through EEG sensors. Configuring, by the at least one processor and at the least one memory, the EEG sensors that are placed along forehead part of the mask for contacting forehead skin to measure human brain activities according to indices of the healthy sleeping time. Configuring, by the at least one processor and at the least one memory, a classification of sleep stages according to real time computing data and modules. Configuring, by the at least one processor and at the least one memory, a thermocouple heating and cooling component for delivering temperature stimulation to skin of wearer of the mask; wherein the thermocouple heating and cooling component is disposed within a physical measurement of boundaries of the sleep mask area after a user wears the sleep mask. Configuring, by the at least one processor and at the least one memory, a computing and electronic gadgets workbench member connected to a thermoregulation member, the EEG electrode sensors, the thermocouple heating and cooling component; wherein a graphical user interface is configured for healthy sleep stages.

The system for a gadget smart sleeping mask collaborating with artificial intelligence machines for configuring, through a human machine interface, a closed loop neuromodulation feedback module for improving sleeping, the system further comprising a Bluetooth system; and a cloud system for configuring a data transfer mechanism.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

For simplicity and clarity of illustration, the drawing figures illustrate the general manner of construction, and description and details of well known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is example of a sleep improving system;

FIG. 2 is another example of a sleep improving system;

FIG. 3 is a schematic of artificial intelligence configurations of a sleep improving system;

FIG. 4 is a still another example of a mask wearer of a sleep improving system;

FIG. 5 is a flow chart of a method of sleep improving;

FIG. 6 is example of a stimulation targets according to a system and method of sleep improving;

FIG. 7 is example of a skin warming on elders according to a system and method of sleep improving;

FIG. 8 is example of a skin warming regarding probabilities of early wakings on elders according to a system and method of sleep improving; and

FIG. 9 is example of a forehead cooling on time to fall asleep on insomniacs according to a system and method of sleep improving.

For simplicity and clarity of illustration, the drawing figures illustrate the general manner of construction, and description and details of well known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide an artificial intelligence system and method health aspects including wellness and fitness. Hereinafter, embodiments of the present disclosure are described in more detail with at least reference to accompanying drawings and regardless of the drawings symbols, same or similar components are assigned with the same reference numerals and thus overlapping descriptions for those are omitted. The suffixes “module” and “unit” for components used in the description below are assigned or mixed in consideration of easiness in writing the specification and do not have distinctive meanings or roles by themselves. In the following description, detailed descriptions of well-known functions or constructions will be omitted since they would obscure the disclosure in unnecessary detail. Additionally, the accompanying drawings are used to help easily understanding embodiments disclosed herein but the technical idea of the present disclosure is not limited thereto. It should be understood that all of variations, equivalents or substitutes contained in the concept and technical scope of the present disclosure are also included.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words “a” or “an”, or other words using the singular or plural number also include the plural or singular number, respectively. Additionally, the words “herein,” “above,” “below,” and words of similar import, when used in this application, shall refer to this application as a whole and not to any particular portions of this application.

The terms “first”, “second”, “third”, “fourth”, and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include”, “have” and any variations thereof, are intended to cover a non-exclusive inclusion, such that a process, method, system, article, device, member, or apparatus that comprises a list of elements is not necessarily limited to those elements, but may include other elements not expressly listed or inherent to such process, method, system, article, device, member, or apparatus.

The terms “left”, “right”, “front”, “back”, “top”, “bottom”, “over”, “under”, and the like in the description and in the claims, if any, are used for descriptive purposes and not necessarily for describing a permanent relative position, for example. It is to be understood that the terms so used are interchangeable under appropriate circumstances such that the embodiments of the invention described herein are, for example, capable of operation in another orientation than those illustrated or another described herein.

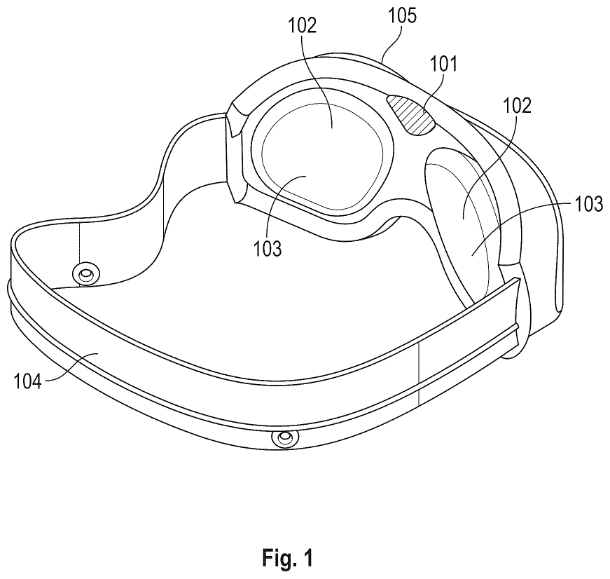

Referring now to FIG. 1, a system and a device 100 are discussed for a gadget smart sleeping mask collaborating with artificial intelligence machines for configuring, through a human machine interface, a closed loop neuromodulation feedback module for improving sleeping. The system including a plurality of EEG electrode sensors 101 connected to a thermoregulation member 102; wherein the EEG sensors 101 are placed along forehead part of the mask 100 for contacting forehead skin of mask wearer to measure human brain activities according to indices of the healthy sleeping time. The thermoregulation member 102 is configured for thermoregulation of facial nerves for enhancing healthy sleeping time. A processor 101 is connected to each of the thermoregulation member 102 and the plurality of the EEG electrodes 101; wherein the processor 101 classifies sleep stages according to real time computing data and modules. A thermocouple heating and cooling component 103 connected to each of the thermoregulation member 102, the plurality of the EEG electrode sensors 101, and the processor 101 for delivering temperature stimulation to skin of wearer of the mask; wherein the thermocouple heating and cooling component 103 is disposed within a physical measurement of boundaries of the sleep mask area after a user wears the sleep mask. A computing and electronic gadgets workbench member 104 connected to each of the thermoregulation member 102, the plurality of the EEG electrode sensors 101, the thermocouple heating and cooling component 103, and the processor 101; wherein a graphical user interface is configured for healthy sleep stages. A back region strap 104 for wearing the mask around the head. A front region 105 of the system is configured for covering the eyes and the head region of the mask wearer. The front region 105 of the sleeping mask 100 covers eyes and region around the eyes; and wherein the mask is fastened, to head of the mask wearer, by a strap around the head, at the back region 104 of the sleeping mask 100. The sleeping mask 100 extends above eyebrows and till hairline of the mask wearer and down below till nostrils of the mask wearer. The thermocouple heating and cooling component 103 comprising a peltier device. The sleeping mask 100 is configured as a sleep wearable medical device 100. A temperature changes module of the sleeping mask 100 is configured to measure temperature changes on skin of face of the mask wearer by affecting autonomous nervous system and brain. The EEG sensors 101 are configured to detect sleep stages. The thermoregulation member 102 stimulates to improve sleep. The thermocouple heating and cooling component 103 is configured as a thermoelectric peltier heater, cooler and conductive gel pad. The system 100 is configured to detect sleep stages & after the processor 101 does the AI optimizations, the system 100 stimulates to improve sleep.

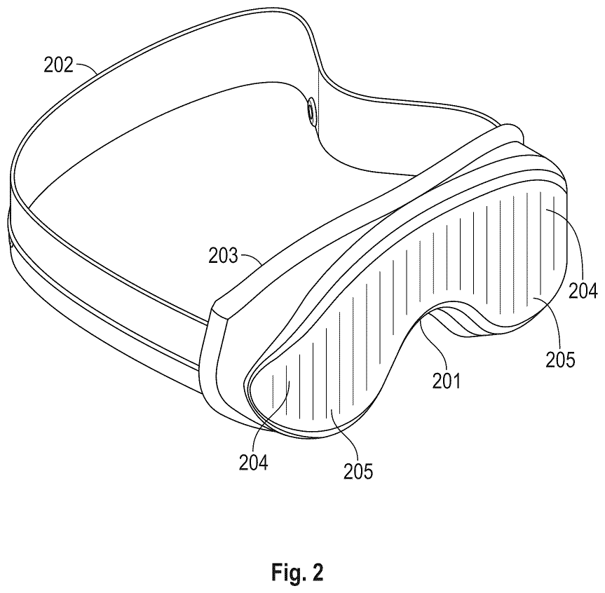

Referring now to FIG. 2, a system and a device 200 are discussed for a gadget smart sleeping mask collaborating with artificial intelligence machines for configuring, through a human machine interface, a closed loop neuromodulation feedback module for improving sleeping. The system including a plurality of EEG electrode sensors 203 connected to a thermoregulation member 204; wherein the EEG sensors 203 are placed along forehead part of the mask 200 for contacting forehead skin of mask wearer to measure human brain activities according to indices of the healthy sleeping time. The thermoregulation member 204 is configured for thermoregulation of facial nerves for enhancing healthy sleeping time. A processor 203 is connected to each of the thermoregulation member 204 and the plurality of the EEG electrodes 203; wherein the processor 203 classifies sleep stages according to real time computing data and modules. A thermocouple heating and cooling component 205 connected to each of the thermoregulation member 204, the plurality of the EEG electrode sensors 203, and the processor 203 for delivering temperature stimulation to skin of wearer of the mask; wherein the thermocouple heating and cooling component 205 is disposed within a physical measurement of boundaries of the sleep mask area after a user wears the sleep mask. A computing and electronic gadgets workbench member 202 connected to each of the thermoregulation member 204, the plurality of the EEG electrode sensors 203, the thermocouple heating and cooling component 205, and the processor 203; wherein a graphical user interface is configured for healthy sleep stages. A back region strap 202 for wearing the mask around the head. A front region 201 of the system is configured for covering the eyes and the head region of the mask wearer. The front region 201 of the sleeping mask 200 covers eyes and region around the eyes; and wherein the mask is fastened, to head of the mask wearer, by a strap around the head, at the back region 202 of the sleeping mask 200 that extends above eyebrows and till hairline of the mask wearer and down below till nostrils of the mask wearer. The thermocouple heating and cooling component 205 comprising a peltier device. The sleeping mask 200 is configured as a sleep wearable medical device 200. A temperature changes module of the sleeping mask 200 is configured to measure temperature changes on skin of face of the mask wearer by affecting autonomous nervous system and brain. The EEG sensors 203 are configured to detect sleep stages. The thermoregulation member 204 stimulates to improve sleep. The thermocouple heating and cooling component 205 is configured as a thermoelectric peltier heater, cooler and conductive gel pad. The system 200 is configured to detect sleep stages & after the processor 203 does the AI optimizations, the system 200 stimulates to improve sleep.

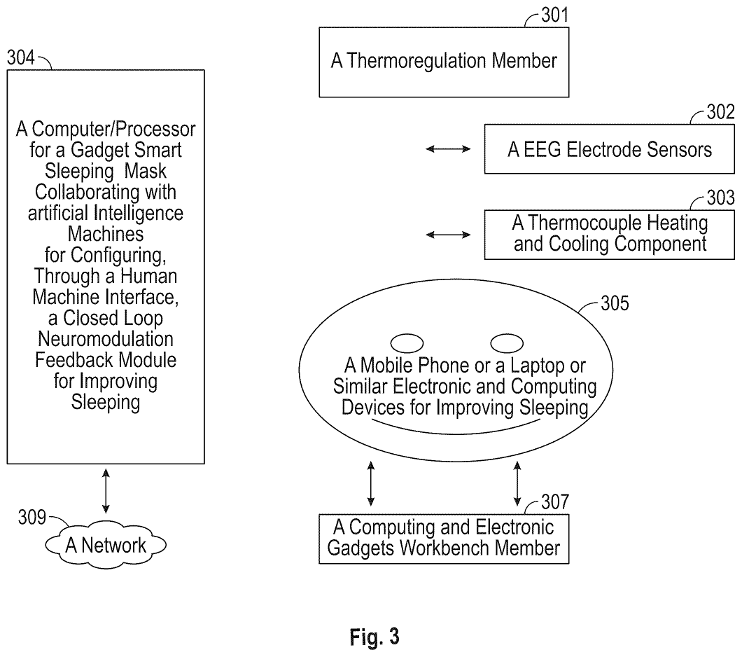

Referring now to FIG. 3, a schematic of artificial intelligence configurations of a sleep improving system 300 after referenced to systems 100 and 200 of FIG. 1 & FIG. 2 respectively, is discussed. The system 301 is a thermoregulation member and the system 302 is a EEG electrode sensor. The system 303 is a thermocouple heating and cooling component. The system 304 is a computer & a processor for a gadget smart sleeping mask collaborating with artificial intelligence machines for configuring, through a human machine interface, a closed loop neuromodulation feedback module for improving sleeping. The system 305 is a mobile phone or a laptop or similar electronic and computing devices for improving sleeping. The system 306 is a database and system 307 is a computing and electronic gadgets workbench member. The system 309 is a network for a wired and a wireless communication among the components of the systems 100, 200, and 300.

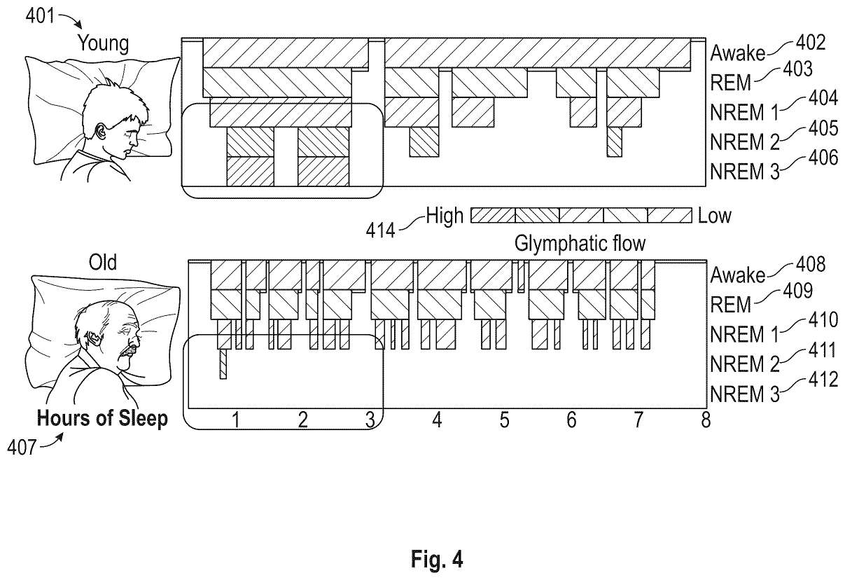

Referring now to FIG. 4, another example of a mask wearer of a sleep improving system 100, 200, and 300 of the FIGS. 1, 2, & 3 respectively is depicted. A young person 401 is depicted using the sleep improving system. A graphical representation 402 depicts awake state of the young person 401; a graphical representation 403 depicts a reminder state of the young person 401 regarding healthy hours of sleeping. A graphical representation 404 depicts a first night reminder state for non rapid eye movement NREM1 for healthy sleeping of the young person 401. A graphical representation 405 depicts a second night reminder state for non rapid eye movement NREM2 for healthy sleeping of the young person 401. A graphical representation 406 depicts a third night reminder state for non rapid eye movement NREM3 for healthy sleeping of the young person 401. An old person 407 is depicted using the sleep improving system. A graphical representation 408 depicts awake state of the old person 407; a graphical representation 409 depicts a reminder state of the old person 407 regarding healthy hours of sleeping. A graphical representation 410 depicts a first night reminder state for non rapid eye movement NREM1 for healthy sleeping of the old person 407. A graphical representation 411 depicts a second night reminder state for non rapid eye movement NREM2 for healthy sleeping of the old person 407. A graphical representation 412 depicts a third night reminder state for non rapid eye movement NREM3 for healthy sleeping of the old person 407. A graphical representation 414 depicts a high transition to a low transition regarding a glymphatic flow parameter of the old person 407 and the young person 401.

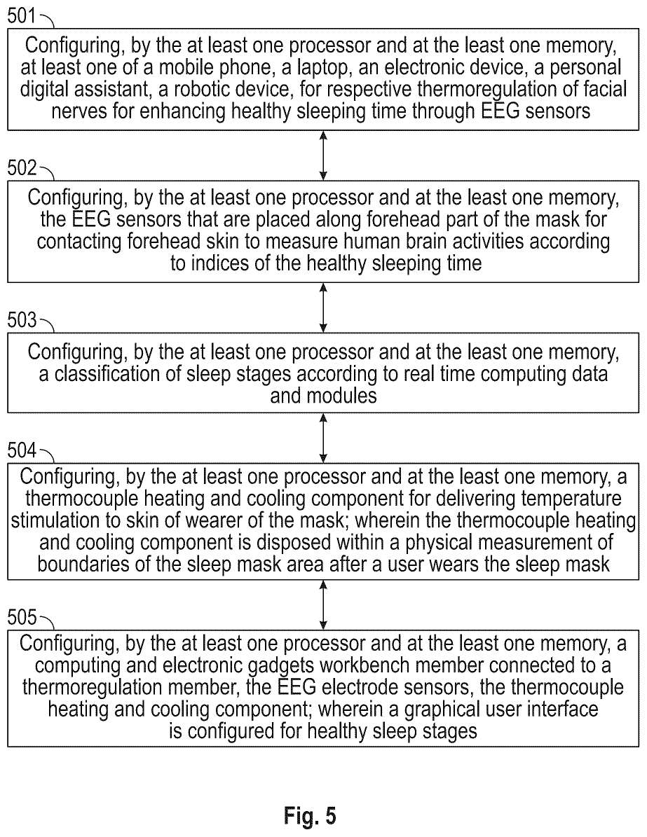

Referring now to FIG. 5, a flow chart of a method of sleep improving is depicted. At a first step 501, the method configures, by the at least one processor and at the least one memory, at least one of a mobile phone, a laptop, an electronic device, a personal digital assistant, a robotic device, for respective thermoregulation of facial nerves for enhancing healthy sleeping time through EEG sensors. At a second step 502, the method configures, by the at least one processor and at the least one memory, the EEG sensors that are placed along forehead part of the mask for contacting forehead skin to measure human brain activities according to indices of the healthy sleeping time. At a third step 503, the method configures, by the at least one processor and at the least one memory, a classification of sleep stages according to real time computing data and modules. At a fourth step 504, the method configures, by the at least one processor and at the least one memory, a thermocouple heating and cooling component for delivering temperature stimulation to skin of wearer of the mask; wherein the thermocouple heating and cooling component is disposed within a physical measurement of boundaries of the sleep mask area after a user wears the sleep mask. At a fifth step 505, the method configures, by the at least one processor and at the least one memory, a computing and electronic gadgets workbench member connected to a thermoregulation member, the EEG electrode sensors, the thermocouple heating and cooling component; wherein a graphical user interface is configured for healthy sleep stages.

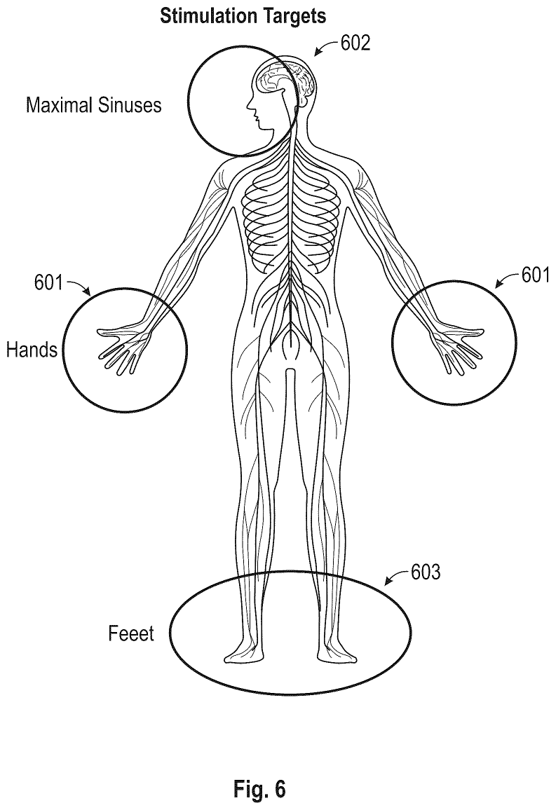

Referring now to FIG. 6, a stimulation targets units of a body of a person using the sleeping system according to the system 100, 200, 300, at least, and method, of sleep improving is depicted. A maximal sinus example regarding hand & regarding feet configurations, of the FIGS. 1, 2, & 3 respectively are depicted. A representation of hands 601 of a person who is sleeping using the system according to the present disclosure is depicted; another representation of maximal sinuses 602 of the head of a person who is sleeping using the system according to the present disclosure is depicted. A representation of feet 603 of a person who is sleeping using the system according to the present disclosure is depicted. These hands, feet, and mind configurations are indicating a healthy sleep standard regarding the sleeping habits of humans, at least.

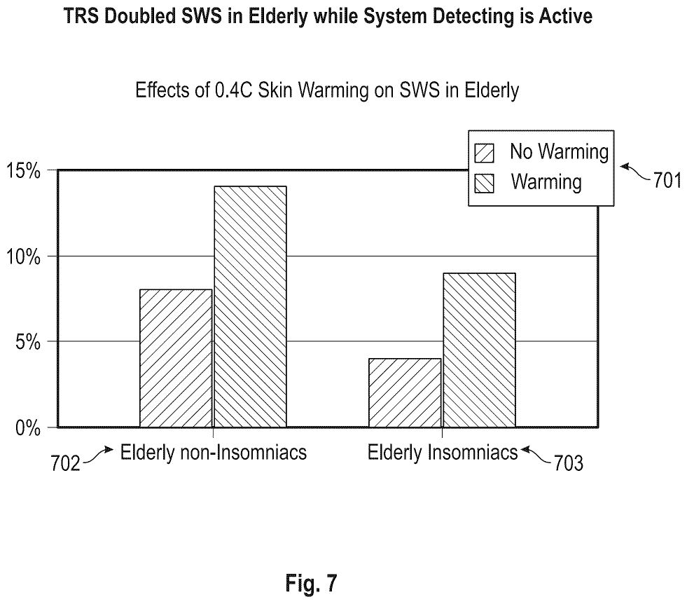

Referring now to FIG. 7, effects of a skin warming on elders according to a system 100, 200, & 300, and a method, of the respective systems of sleep improving, according to this disclosure, is depicted. A graphical representation 701 depicts warming and no warming effects at the sleeping person. A graphical representation 702 indicates effects of warming and no warming at elderly non insomniacs; a graphical representation 703 indicates effects of warming and non warming at elderly insomniacs. The system 100, 200, and 300 of the present disclosure are configured to detect the graphical effects depicted in the FIG. 7 that for example relates to TRS doubled Slow Wave Sleep (SWS) sometimes known as deep sleep.

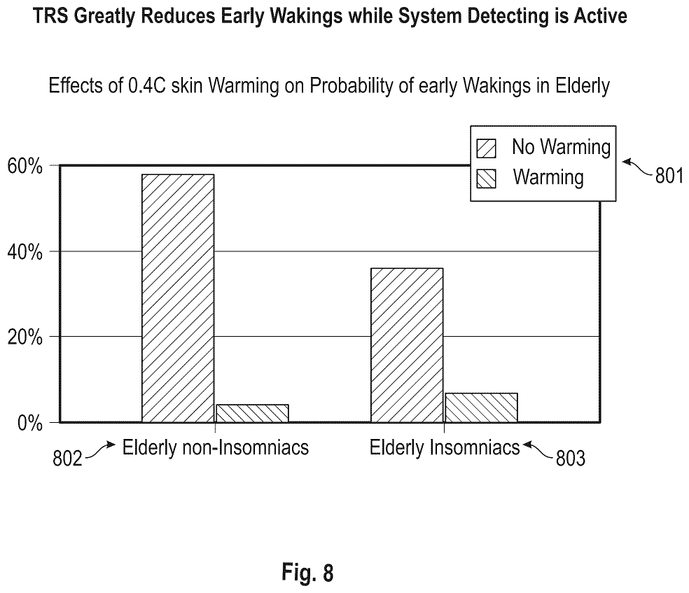

Referring now to FIG. 8, effects of a skin warming regarding probabilities of early wakings on elders according to a system 100, 200, & 300, and method, of the respective systems of sleep improving, according to this disclosure is depicted. A graphical representation 801 depicts warming and no warming effects at the sleeping person. A graphical representation 802 indicates effects of warming and no warming at elderly non insomniacs; a graphical representation 803 indicates effects of warming and non warming at elderly insomniacs. The system 100, 200, and 300 of the present disclosure are configured to detect the graphical effects depicted in the FIG. 8 that for example relates to Treatment Resistant Schizophrenia (TRS) that mostly reduces early wakings.

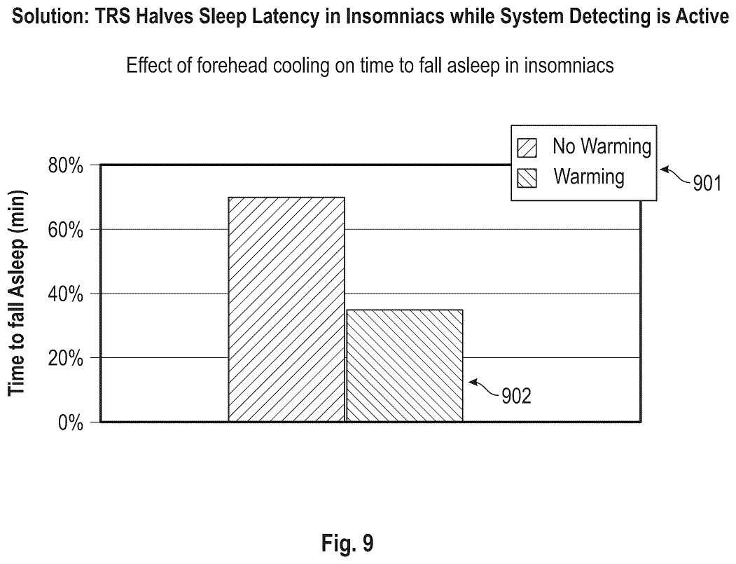

Referring now to FIG. 9, effects of a forehead cooling on time to fall asleep on insomniacs according to a system 100, 200, & 300, and method of the respective systems of sleep improving, according to this disclosure is depicted. A graphical representation 801 depicts warming and no warming effects at the sleeping person. A graphical representation 802 indicates effects of sleep insomniacs regarding the configuration in which the systems 100, 200, & 300 regulate the effect of forehead cooling on time to fall asleep in insomniacs. The system 100, 200, and 300 of the present disclosure are configured to detect the graphical effects depicted in the FIG. 9 that for example relates to Treatment Resistant Schizophrenia (TRS) that halves sleep latency in insomniacs.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. For instance, although the disclosed generator is preferred to be installed in a vehicle, other applications may be realized without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

Replacement of one or more claimed elements constitutes a reconstruction and not repair. According to another example, benefits, other advantages, and solutions to problems have been described with regard to specific embodiments. The benefits, the advantages, the solutions to problems, and any element or elements that may cause any benefit, any advantage, or any solution to occur or become more pronounced, however, are not to be construed as any critical, any required, or any essential features or any elements of any or all the claims, unless such benefits, advantages, solutions, or elements are expressly stated in such a claim.

As it is depicted in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

Also, what has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for a gadget smart sleeping mask collaborating with artificial intelligence machines for configuring, through a human machine interface, a closed loop neuromodulation feedback module for improving sleeping, the system comprising:
a thermoregulation member configured for thermoregulation of facial nerves for enhancing healthy sleeping time;
a plurality of EEG electrode sensors connected to the thermoregulation member;
wherein the EEG sensors are placed along forehead part of the mask for contacting forehead skin to measure human brain activities according to indices of the healthy sleeping time;
a processor connected to each of the thermoregulation member and the plurality of the EEG electrodes; wherein the processor classifies sleep stages according to real time computing data and modules;
a thermocouple heating and cooling component connected to each of the thermoregulation member, the plurality of the EEG electrode sensors, and the processor for delivering temperature stimulation to skin of wearer of the mask; wherein the thermocouple heating and cooling component is disposed within a physical measurement of boundaries of the sleep mask area after a user wears the sleep mask; and
a computing and electronic gadgets workbench member connected to each of the thermoregulation member, the plurality of the EEG electrode sensors, the thermocouple heating and cooling component, and the processor; wherein a graphical user interface is configured for healthy sleep stages.

2. The system of claim 1, wherein a front region of the sleeping mask covers eyes and region around the eyes; and wherein the mask is fastened, to head of the mask wearer, by a strap around the head, at a back region of the sleeping mask.

3. The system of claim 1, wherein the sleeping mask extends above eyebrows and till hairline of the mask wearer and down below till nostrils of the mask wearer.

4. The system of claim 1, wherein the thermocouple heating and cooling component comprising a peltier device.

5. The system of claim 1, wherein the sleeping mask is configured as a sleep wearable medical device.

6. The system of claim 1, further comprising a temperature changes module configured to measure temperature changes on skin of face of the mask wearer by affecting autonomous nervous system and brain.

7. A gadget smart sleeping mask device collaborating with artificial intelligence machines for configuring, through a human machine interface, a closed loop neuromodulation feedback module for improving sleeping, the device comprising:
a thermoregulation member configured for thermoregulation of facial nerves for enhancing healthy sleeping time;
a plurality of EEG electrode sensors connected to the thermoregulation member; wherein the EEG sensors are placed along forehead part of the mask for contacting forehead skin to measure human brain activities according to indices of the healthy sleeping time;
a processor connected to the thermoregulation member and the plurality of the EEG electrodes; wherein the processor classifies sleep stages according to real time computing data and modules;
a thermocouple heating and cooling component connected to the thermoregulation member, the plurality of the EEG electrode sensors, and the processor for delivering temperature stimulation to skin of wearer of the mask; wherein the thermocouple heating and cooling component is disposed within a physical measurement of boundaries of the sleep mask area after a user wears the sleep mask; and
a computing and electronic gadgets workbench member connected to the thermoregulation member, the plurality of the EEG electrode sensors, the thermocouple heating and cooling component, and the processor; wherein a graphical user interface is configured for healthy sleep stages.

8. A computer implemented method for a sleeping mask device that configures, through a human machine interface, a closed loop neuromodulation feedback module for improving sleeping, the method comprising:
configuring, by the at least one processor and at the least one memory, at least one of a mobile phone, a laptop, an electronic device, a personal digital assistant, a robotic device, for respective thermoregulation of facial nerves for enhancing healthy sleeping time through EEG sensors;
configuring, by the at least one processor and at the least one memory, the EEG sensors that are placed along forehead part of the mask for contacting forehead skin to measure human brain activities according to indices of the healthy sleeping time;
configuring, by the at least one processor and at the least one memory, a classification of sleep stages according to real time computing data and modules;
configuring, by the at least one processor and at the least one memory, a thermocouple heating and cooling component for delivering temperature stimulation to skin of wearer of the mask; wherein the thermocouple heating and cooling component is disposed within a physical measurement of boundaries of the sleep mask area after a user wears the sleep mask; and
configuring, by the at least one processor and at the least one memory, a computing and electronic gadgets workbench member connected to a thermoregulation member, the EEG electrode sensors, the thermocouple heating and cooling component; wherein a graphical user interface is configured for healthy sleep stages.

9. The system for a gadget smart sleeping mask collaborating with artificial intelligence machines for configuring, through a human machine interface, a closed loop neuromodulation feedback module for improving sleeping, of claim 1, the system further comprising a Bluetooth system; and a cloud system for configuring a data transfer mechanism.
